(19) United States
(12) Patent Application Publication
(10) Pub. No.: US 2018/0233224 A1
(43) Pub. Date: Aug. 16, 2018

(54) RADIOLOGY IMAGE SEQUENCING FOR OPTIMAL READING THROUGHPUT

(71) Applicant: KONINKLIJKE PHILIPS N.V., EINDHOVEN (NL)

(72) Inventor: KAREN IRENE TROVATO, PUTNAM VALLEY, NY (US)

(73) Assignee: KONINKLIJKE PHILIPS N.V., EINDHOVEN (NL)

(21) Appl. No.: 15/751,203

(22) PCT Filed: Aug. 3, 2016
(86) PCT No.: PCT/IB2016/054661

§ 371 Date: Feb. 8, 2018

Related U.S. Application Data
(60) Provisional application No. 62/208,857, filed on Aug. 24, 2015.

(51) Int. Cl. G16H 40/20; G06F 17/30; G16H 30/20

(52) U.S. Cl. CPC G16H 40/20; G16H 30/20; G06F 17/3028

(57) ABSTRACT

A device operating in conjunction with a Picture Archiving and Communication System (PACS) (10) stores a plurality of radiological images, which includes at least two different image modalities. The device includes a radiology workstation (14) with at least one display device (20, 22). An electronic processor (36) is programmed to: organize a queue (32) of radiology examination reading tasks in accord with information (60, 62) about the radiology examination reading tasks other than or in addition to their order in the queue to generate an ordered work list (34) of the radiology examination reading tasks; display the ordered work list on the at least one display device of the radiology workstation; and retrieve from the PACS one or more radiology images of a radiology examination reading task of the ordered work list and display the retrieved radiology images on the at least one display device of the radiology workstation.

### FIELD

The following relates generally to the radiology arts, radiology reading arts, medical picture archiving and communications system (PACS) arts, radiology workstation arts, radiology workstation user interfacing arts, and related arts.

### BACKGROUND

Radiologists are highly specialized medical professionals, and as such are expected to maintain a high throughput. In a typical work environment, the radiologist is seated at a PACS workstation running radiology workstation software such as the Philips iSite PACS workstation system (available from Koninklijke Philips N.V., Eindhoven, the Netherlands). A queue is maintained listing the radiology reading tasks to be performed by the radiologist (or team of radiologists) for that work shift. The radiologist selects a next task to perform from the queue, reads the images, and dictates a report of findings (i.e., the radiology report), which is sent to the patient's physician and also stored on the PACS.

Each radiology reading task typically has a compensation value designated by Relative Value Units (RVUs). For example, in some institutions, a CT reading is assigned 4 RVU points, an MRI reading is assigned 8 RVU points, and computed radiography (i.e., an x-ray) reading is assigned 1 RVU point. Other imaging modalities, such as positron emission tomography (PET) images or a single photon emission computed tomography (SPECT) images, can have their own RVU values. The radiologist is expected to perform readings with a certain number of total RVU points per shift. In some medical institutions, RVU points are assigned based on a corresponding medical procedure code, as these codes are used for billing. Two common medical procedure coding systems are Current Procedural Terminology (CPT) codes and Healthcare Common Procedure Coding System (HCPCS) codes. In the case of medical imaging procedures, the procedure codes are delineated by imaging modality, anatomical region, and perhaps other features such as clinical task. Radiologists typically read between 3200 to over 6000 ‘RVU points’ per year, calling for a high level of efficiency.

The queue of radiology examination reading tasks is conventionally ordered by time of entry into the queue. The radiologist either works through the work list in order (“first in, first out”), which may not be the best order, or cherry-picks the next task which takes extra time as the radiologist must skim through the list and make the next selection based on the limited information available for each reading task in the work list.

### BRIEF SUMMARY

In accordance with one illustrative example, a device operating in conjunction with a Picture Archiving and Communication System (PACS) stores a plurality of radiological images, which includes at least two different image modalities. The device includes a radiology workstation with at least one display device. An electronic processor is programmed to: organize a queue of radiology examination reading tasks in accord with information about the radiology examination reading tasks other than or in addition to their order in the queue to generate an ordered work list of the radiology examination reading tasks; display the ordered work list on the at least one display device of the radiology workstation; and retrieve from the PACS one or more radiology images of a radiology examination reading task of the ordered work list and display the retrieved radiology images on the at least one display device of the radiology workstation.

In accordance with another illustrative example, a device operating in conjunction with a Picture Archiving and Communication System (PACS) includes a radiology workstation with at least one display device and at least one user input device. An electronic processor is programmed to: retrieve a queue of radiology examination reading tasks in which the reading tasks are ordered by time of entry into the queue; organize the queue of radiology examination reading tasks in accord with reading task features including or derived from at least one of imaging modality, imaged anatomy, radiology examination type, and examination subject to generate an ordered work list of the radiology examination reading tasks; display the ordered work list on the at least one display device of the radiology workstation; receive a selection of a radiology examination reading task from the ordered work list via the at least one user input device of the radiology workstation; and retrieve from the PACS one or more radiology images of the selected radiology examination reading task and display the retrieved radiology images on the at least one display device of the radiology workstation.

One advantage resides in providing a radiology workstation with a more efficient user interface.

Another advantage resides in providing a radiology workstation providing for more efficient allocation of radiology examination reading tasks to one or more radiologists.

Further advantages of the present invention will be appreciated to those of ordinary skill in the art upon reading and understand the following detailed description. It will be appreciated that a given embodiment may provide none, one, two, or more of these advantages.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a radiology workstation as disclosed herein.

FIG. 2 diagrammatically illustrates an input table for the radiology workstation of FIG. 1.

FIG. 3 diagrammatically illustrates a first work list displayed by the radiology workstation of FIG. 1.

FIG. 4 diagrammatically illustrates a first reading schedule work list generated by the work list of FIG. 3.

FIG. 5 diagrammatically illustrates a second work list displayed by the radiology workstation of FIG. 1.

FIGS. 6 and 7 are graphs showing data related to reading time vs. actual time of images by a radiologist.

FIG. 8 shows a work schedule generated based on the data from FIGS. 6 and 7.

### DETAILED DESCRIPTION

Radiology examination reading tasks are queued for reading, usually in the order of arrival. Some radiology departments employ a “first in, first out” workflow, in which radiology reading tasks are performed in the order they arrive (that is, in the order of the queue). However, this approach can overstress a radiologist if, for example, the radiologist is forced to perform several complex and mentally taxing readings in a row due to their arrival order in the queue. To reduce the stress level, many radiology departments permit the radiologist to choose the next reading task to perform from the queue of radiology examination reading tasks. This allows the radiologist to interleave difficult and easier reading tasks in order to reduce stress, or otherwise organize the reading tasks to the radiologist's preferences. Reduced radiologist stress is expected to lead to more accurate readings, and ultimately to higher efficiency.

However, the selection of reading tasks takes valuable time, and furthermore the quasi-random sequencing (i.e. by order of arrival) of reading tasks in the queue may make it difficult for the radiologist to identify the “best” next reading task to perform. For example, the radiologist may wish to perform several reading tasks of the same anatomy in a row, but may find it difficult to locate all such reading tasks by visually scanning the queue. Likewise, if the queue includes two or more radiology examination reading tasks for the same radiology examination subject (e.g. a CT and MRI of the same patient) it might be advantageous to perform these together, so as to leverage information from the different examinations but it may be difficult for the radiologist to identify this set of reading tasks for the same patient.

Embodiments disclosed herein organize the queue into a work list having a principled ordering of reading tasks. The organizing is in accord with information about the radiology examination reading tasks other than or in addition to their order in the queue. For example, the organization may utilize reading task features including or derived from at least one of: imaging modality; imaged anatomy; radiology examination type; and examination subject.

In one approach, a work schedule is defined which delineates scheduled time blocks of a work shift (or work day) allocated to designated reading types. Thus, for example, the first half-hour may be assigned to computed radiography (CR) or direct radiography (DR) readings which are relatively easy and serve as a “warm-up” period, followed by a scheduled time block dedicated to more complex readings (e.g. MR or CT), and so forth. Within each scheduled time block, the ordering of the reading tasks assigned to that block may be by time of entry into the queue, or may be ordered on some other basis. The number of reading tasks that fit into one scheduled time block is estimated based on estimated reading times for the tasks. Since all tasks of the work list usually must be completed over the course of the work shift or work day, one or more time blocks are allocated to general or mixed use. With the scheduled time blocks defined (optionally in manual fashion by the radiologist similar to an electronic calendar scheduling), the system classifies tasks by reading type and allocates tasks into time blocks accordingly. In a variant of this approach, data mining is performed on the past work history of a radiologist to identify the time blocks.

In another approach, a set of rules is applied to order reading tasks of the work list. Two illustrative rules are as follows: Rule 1: group reading tasks for a single patient (more generally, radiology examination subject) together; and Rule 2: group similar reading tasks together. The rationale for Rule 1 is that it facilitates the radiologist developing a holistic understanding of the patient's overall condition and immediate past images, saving time. The rationale for Rule 2 is that efficiency is improved by limiting abrupt context switches between successive reading tasks. For Rule 2, automated task type classification is again applied.

These approaches may be variously combined in some embodiments. For example, scheduled time blocks may be assigned to different examination types and, within each time block tasks may be automatically grouped by patient and/or examination similarity. In the case of a patient with multiple reading tasks of very different types, these may be grouped in a general or mixed use time block.

As used herein, a “patient” refers to a radiology examination subject (or “examination subject” for brevity). The term “patient” as used herein broadly encompasses hospital in-patients, hospital out-patients, emergency room patients, independent imaging center clients, persons who visit a medical office of any kind and are directed to a radiology facility for a radiology examination, or so forth.

The term “Picture Archiving and Communication System” or “PACS” as used herein broadly encompasses any electronic database that stores radiology images acquired during radiology examinations and provides retrieval access for the stored radiology images. The PACS is distinct from general-purpose medical databases such as the Electronic Medical Record (EMR) or Electronic Health Record (EHR), although some integration of the PACS with a general-purpose medical database is contemplated. For example, the patient record in the EMR or EHR may include hyperlinks to radiology examinations stored in the PACS, and/or the PACS record for a patient may include a hyperlink to the patient's record in the EMR or EHR. In typical embodiments, the PACS stores radiology images in accordance with the Digital Imaging and Communications in Medicine (DICOM) file format definition promulgated by the National Electrical Manufacturers Association (NEMA), or in a variant of the standard DICOM definition.

Information, including current examination information can be stored for a current radiology examination, such as the reason for examination, the imaging modality of the examination, and/or the number of RVU points for the examination. The reason for examination is typically indicated by the ordering physician, and is commonly (though not necessarily) stored as an International Classification of Diseases (ICD-9) code which is a standard classification system used by medical institutions, medical insurance companies, and the like. The imaging modality may be obtained from the examination metadata or from metadata of individual images. For example, the standard DICOM header includes a field for specifying the image modality. The RVU points are generally a function of imaging modality and possibly ICD-9 code, and hence can be calculated. Other metadata of the current radiology examination and/or the DICOM headers of the images may also be used in organizing tasks of the radiology examination, such as the examination date, the number of images in the examination, image size/resolution, or so forth.

Some basic patient demographic information is also stored in the PACS. This is the demographic information for the examination subject of the selected radiology examination reading task. Such data generally include at least sex and date of birth, and may also include data such as ethnicity.

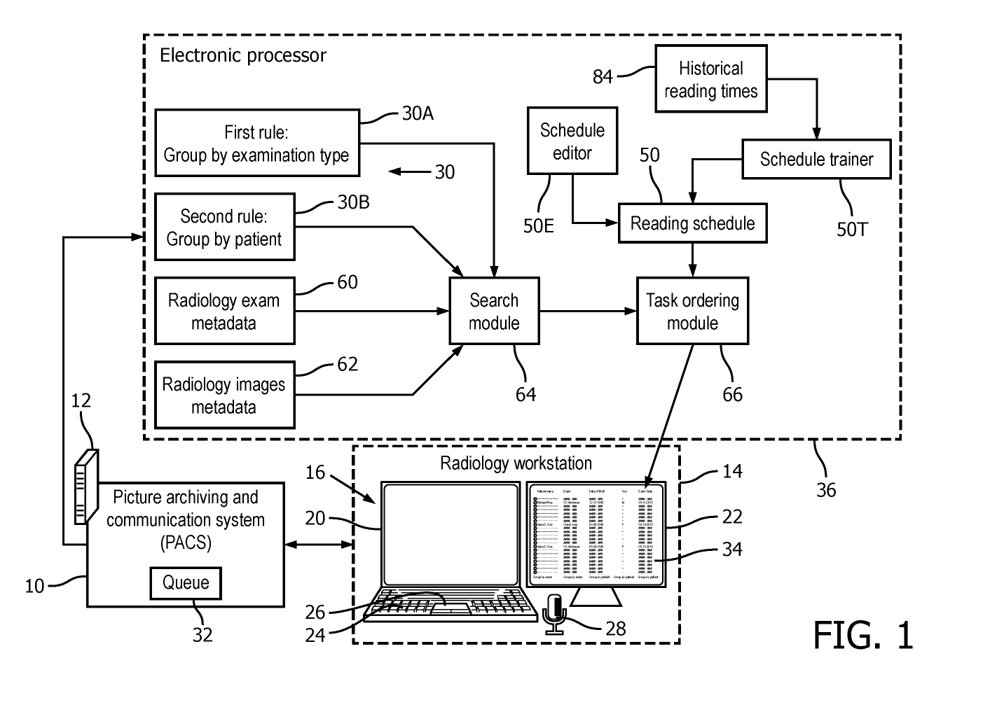

With reference to FIG. 1, a Picture Archiving and Communication System (PACS) 10 is implemented on a networked computing system 12 diagrammatically indicated in FIG. 1 by a server computer. It will be appreciated that the networked computing system 12 may comprise a single server computer, a computing cluster, a cloud computing resource, or so forth. The PACS 10 installed on the networked computing system 12 is connected with one or (more typically) a plurality of radiology workstations, where FIG. 1 illustrates a single representative radiology workstation 14, via a secure electronic data network, such as a wired and/or wireless Wide Area Network (WAN) implemented via Ethernet, WiFi, or another suitable wired and/or wireless electronic data networking protocol. The secure electronic data network should have sufficient bandwidth to communicate radiology images, which are typically large data files, to and from the radiology workstation 14. Optionally, the PACS 10 installed on the networked computing system 12 may be connected with other computing systems such as physician's desktop computers, radiological imaging system controllers (e.g. MRI or CT system controllers) or so forth (not shown).

Each radiology workstation 14 includes an electronic processor, for example embodied as a computer 16. Each radiology workstation 14 further includes at least one display device, e.g. an illustrative display device 20 of the computer 16 and an additional display device 22. This display device(s) 20 or 22 may include a browser. Providing the radiology workstation 14 with two (or more) display devices 20, 22 can be advantageous as it allows one display device to be used to display textual content or other auxiliary information while the other display device is used as a dedicated radiology image viewer; however a radiology workstation with only a single display device is also contemplated. At least one display device of the radiology workstation should be a high-resolution display capable of displaying radiology images with sufficiently high resolution to enable the radiologist to accurately read the radiology image. Each radiology workstation 14 further includes at least one user input device, such as: an illustrative computer keyboard 24; a mouse, touchpad 26, or other pointing device; a touch-sensitive display (e.g., one or both display devices 20, 22 may be a touch-screen display); a dictation microphone 28, or so forth. Optionally, the radiology workstation 14 is further capable of measuring a reading time defined between selection of a radiology examination reading task and completing receipt of the entry of the radiology report for that task with a timer (not shown) implemented by the computer 16, e.g. using the internal (i.e. system) clock of the computer.

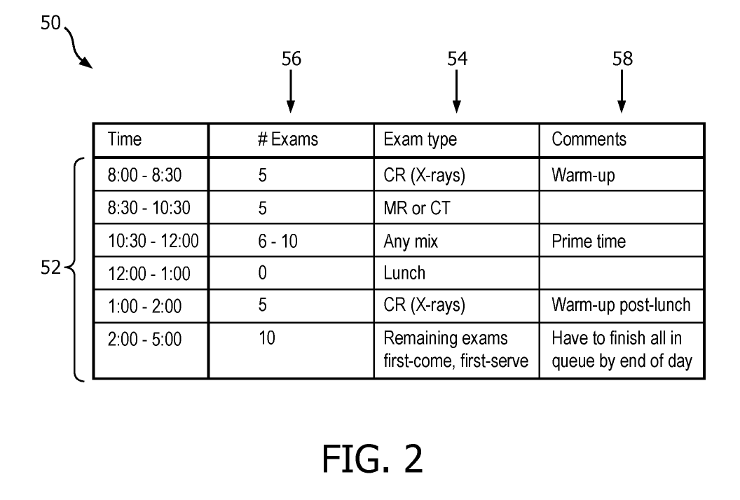

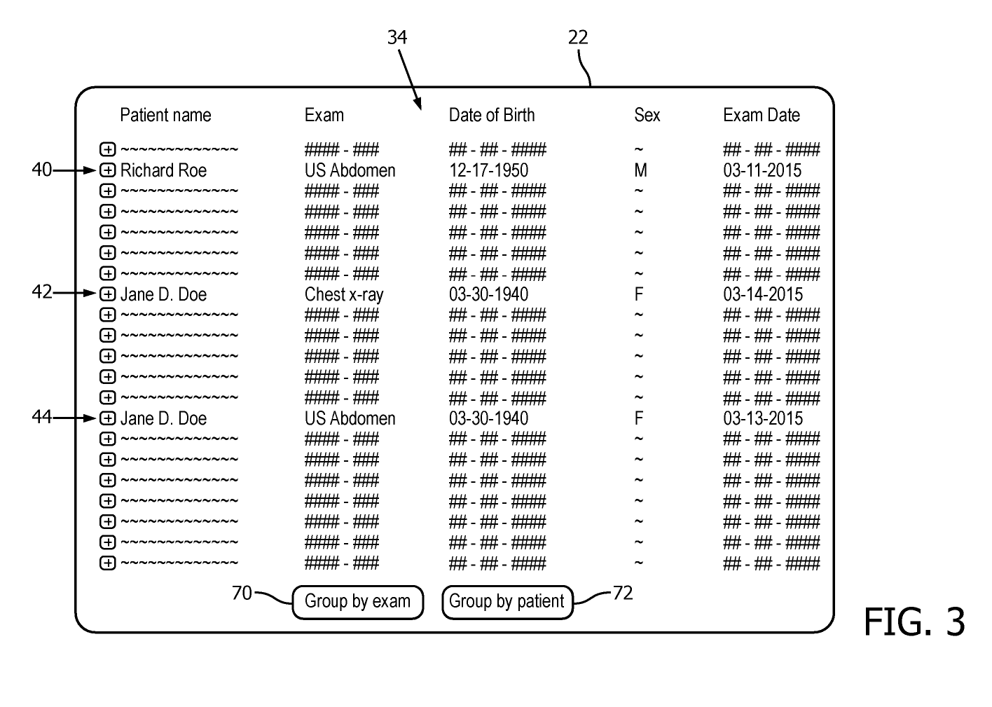

With continuing reference to FIG. 1 and with further reference to FIGS. 2 and 3, the radiology workstation 14 operating in conjunction with the PACS 10 installed on the networked computing system 12, provides a work environment for a radiologist as follows. One or more rules 30 are used to generate and organize a queue 32 of radiology examination reading tasks. The queue 32 is a list of radiology examination reading tasks that have not yet been performed (i.e. for which a radiology report has not yet been entered or stored in the PACS 10). The queue is usually organized by arrival time, i.e. the first reading task to arrive is at the top of the queue 32, and the most recently arrived reading task is at the bottom of the queue 32. The illustrative queue 32 is maintained on the PACS 10 which is convenient in the case of a larger radiology department that may have two or more radiologists working a single shift via two or more instances of the illustrative radiology workstation 14 in this arrangement the same queue 32 is then accessed by each radiologist so that they can mutually track remaining reading tasks. Alternatively, it is contemplated for the queue 32 to be maintained at the radiology workstation, which may be appropriate in a setting in which only a single radiology workstation 14 is provided. An electronic processor 36 is programmed to organize the queue 32 of radiology examination reading tasks in accord with information about the radiology examination reading tasks other than or in addition to their order in the queue to generate an ordered work list 34 of the radiology examination reading tasks. The electronic processor 36 may be a component of the radiology workstation 14, e.g. implemented by suitably programming the computer 16, or the electronic processor 36 may be a component of the PACS 10, e.g. implemented by suitably programming the server computer 12.

The queue 32 is displayed as the ordered work list 34 on a display device of the radiology workstation. In illustrative FIGS. 1 and 3, the ordered work list 34 is displayed on the computer display device 22, although in other embodiments it might be displayed on the display device 20, or the radiology workstation 14 may optionally be configured to display the work list display 34 on a selectable one of the display devices 20, 22. The illustrative ordered work list 34 shows, for each radiology examination reading task, a number of data fields identified by respective headings: “Patient name”, “Exam(ination type)”, “Date of Birth”, “Sex”, and “Exam(ination) Date (and time)”. Although not shown, additional or other fields may be displayed, such as an MRN field (where “MRN” stands for “Medical Record Number”, or equivalently, PatientID) or an Accession number field. Accession number refers to the current image(s), typically of the same modality taken at the same imaging event. These are merely illustrative data fields, and additional or other data fields are contemplated to be displayed in the display of the work list 34. For illustrative purposes, the displayed ordered work list 34 shown in FIG. 3 includes three illustrative radiology examination reading tasks: a reading task 40 for patient “Richard Roe”; a reading task 42 for patient “John J. Smith”; and a reading task 44 for patient “Jane D. Doe”. The remaining illustrative radiology examination reading tasks of the display 32D are diagrammatically indicated using placeholder symbols “˜” (tilde) and “#” (pound sign).

A radiologist can select the ordering of the tasks of the ordered work list 34 based on one or more rules 30. (Alternatively, these rules may be hard-coded and not selectable by the radiologist). With continuing reference to FIG. 1 and with further reference to FIGS. 2 and 3, the radiologist can select an organizational order of the reading tasks of the work list 34 with a first rule 30A that groups reading tasks by radiology examination type, and a second rule 30B that groups reading tasks by patient.

With particular reference to FIG. 2, in some embodiments the work list 34 is constructed at least in part on the basis of a reading schedule 50 which can include one or more time blocks 36 related to reviewing only CR images, only MR images, only CT images, only PET images, only SPECT images, or mixture including at least one types of these different image modalities. For example, the reading schedule 50 may be displayed on the display 20 or 22, and a schedule editor 50E (which may, for example, be a web-based spreadsheet editor) enables the radiologist to define the time blocks 52 with the one of the user input devices (e.g., the keyboard 24; the mouse or touchpad 26, or the microphone 28). Each time block 52 is defined in terms of an examination type 54 to be read during that time block, the number of examinations 56 that can be suitably conducted during the time block, preferably computed automatically by dividing the time duration of the time block by the average reading time for an examination of the designated exam type 54, or entered manually, and an optional comments section 58. FIG. 2 shows an illustrative example of the reading schedule 50 having: a time block 8-8:30 am for CR or x-ray examinations (which are relatively simple and hence serve as a “warm-up” period); a time block 8:30-10:30 for MRI or CT examinations; a time block of 10:30-12 noon with no limitations on the examination type; a one-hour lunch break; a time block 1-2 pm for CR examinations; and a final time block of 2-5 pm with no examination type limitations that ensures time is available to complete all reading tasks of the shift.

The queue 32 is then organized into the ordered work list 34 by assigning reading tasks to time blocks 52 of the reading schedule 50 based on the reading tasks having a time block-defining feature (e.g. the examination type 54 in illustrative FIG. 2). In other contemplated examples, the time block-defining feature may be an imaging modality, a radiology examination type, an imaged anatomy, or a combination thereof. Appropriate reading tasks are assigned to a given time block until it is “filled”, that is, until the total expected reading time for the assigned tasks fills the duration of the time block. If not enough reading tasks having the block-defining feature are in the queue 32, then the remaining time can be filled with tasks of another type, or the next-adjacent time block can be expanded to fill the available time, or some other remedial action can be taken.

With continuing reference to FIG. 2 and with reference back to FIG. 1, reading tasks are assigned to time blocks based on information about the radiology examination reading tasks other than or in addition to their order in the queue 32. In illustrative FIG. 1, a schedule editor 50E operating in conjunction with suitable user interfacing devices (e.g., the devices 24, 26 of the workstation 14) enables the radiologist to create the schedule 50, e.g. by defining the time blocks 52 and the block-defining features 54. Appropriate reading task features are identified for the various reading tasks of the queue 32 and are used for such assignments. A reading task feature may, by way of illustration, include or be derived from imaging modality, imaged anatomy, radiology examination type, and examination subject, or so forth. These reading task features may be identified from metadata 60 associated with the radiology examination and/or from metadata 62 associated with the radiology images of the radiology examination. A search module 64 searches the reading tasks of the queue 32 to group reading tasks with the block-defining task feature (using the examination type-grouping rule 30A in the illustrative example), and a task ordering module 66 then orders the reading tasks of the queue 32 in accord with this information by assigning appropriate tasks to time blocks 52 of the reading schedule 50 to generate the work list 34.

The radiologist viewing the displayed ordered work list 34 chooses a reading task from the work list 34 within the appropriate time block of the reading schedule 50, e.g. using at least one user input device 24, 26, 28. Upon selection, the radiology workstation 14 retrieves one or more radiology images of the selected radiology examination reading task from the PACS 10 and displays the retrieved radiology images, e.g. on the display device 20. This display may incorporate usual image display or rendering techniques such as zoom, pan, resizing, displaying selected images side-by-side or in another arrangement, allowing the radiologist to use on-screen cursors to perform spatial and/or intensity measurements, or so forth. It will be appreciated that only one image, or a subset of a set of images, or all images, may be displayed at any given time during the reading process. For example, the radiologist may choose to work through a set of image slices one-by-one so that only a single image slice is displayed at any given time. Optionally, the radiologist may bring up and display images from other radiology examinations, e.g. to compare a current tumor image with one acquired in an earlier radiology examination to observe growth or shrinkage of the tumor. During the reading, the radiology workstation 14 receives, via the at least one user input device, entry of a radiology report for the selected radiology examination reading task. In a common approach, the dictation microphone 28 is used to receive entry of an orally dictated radiology report; however, it is additionally or alternatively contemplated to employ another user input device, such as using the keyboard 24 to type in the radiology report or to edit the initially orally dictated report. When the radiologist is satisfied with the entered radiology report for the selected radiology examination reading task, the radiologist performs suitable operations to save the report in the PACS 10, send the report to the patient's physician, or otherwise store and/or disseminate the report. For example, the radiology workstation 14 may display a “file report” button or the like which can be selected by the radiologist using a pointer or the like to execute the filing of the report. The work list 34 (and the underlying queue 32) is updated by removing the completed radiology examination reading task from the queue 32 and work list 34, and the updated work list 34 is displayed on the radiology workstation 14. Unless up to a break, the radiologist will then move on to select a next radiology examination reading task to perform as just described.

Because the work list 34 is organized according to the reading schedule 50, the topmost reading task on the work list 34 is generally expected to be an appropriate choice for selection as the next reading task. In some cases, the radiologist may select some other reading task other than the topmost reading task, but typically it will still be a task within the current time block of the reading schedule 50. In a variant embodiment, the radiologist may be required to select the topmost reading task of the work list 34, or may be required to select a reading task assigned to the current time block of the reading schedule 50. In the case of the latter, one way to enforce this requirement is to display only those reading tasks assigned to the current time block in the displayed (portion of) the work list. However, it is understood that ‘STAT exams’, ultra-high priority determining life and death, may break into any schedule created. Once the STAT exam is read, the prior schedule can resume.

In some embodiments, the reading schedule 50 is not employed, and instead the electronic processor 36 organizes the queue 32 of radiology examination reading tasks in accord with rules 30 to generate the ordered work list 34. In such embodiments, there are no defined time blocks.

With reference now to FIGS. 3-5, some illustrative embodiments that do not employ the reading schedule 50 are described. FIG. 3 again shows the displayed ordered work list 34 including three illustrative radiology examination reading tasks: a reading task 40 for patient “Richard Roe”; a reading task 42 for patient “John J. Smith”; and a reading task 44 for patient “Jane D. Doe”. The remaining illustrative radiology examination reading tasks of the display 32D are diagrammatically indicated using placeholder symbols “” (tilde) and “#” (pound sign). As further shown in FIG. 3, the radiologist can select which of the rules 30A, 30B are used to organize the queue 32 into the work list 34. In illustrative FIG. 3, this is done by selecting a “Group by exam” button 70 to apply rule 30A, or by selecting a “Group by patient” button 72 to apply rule 30B.

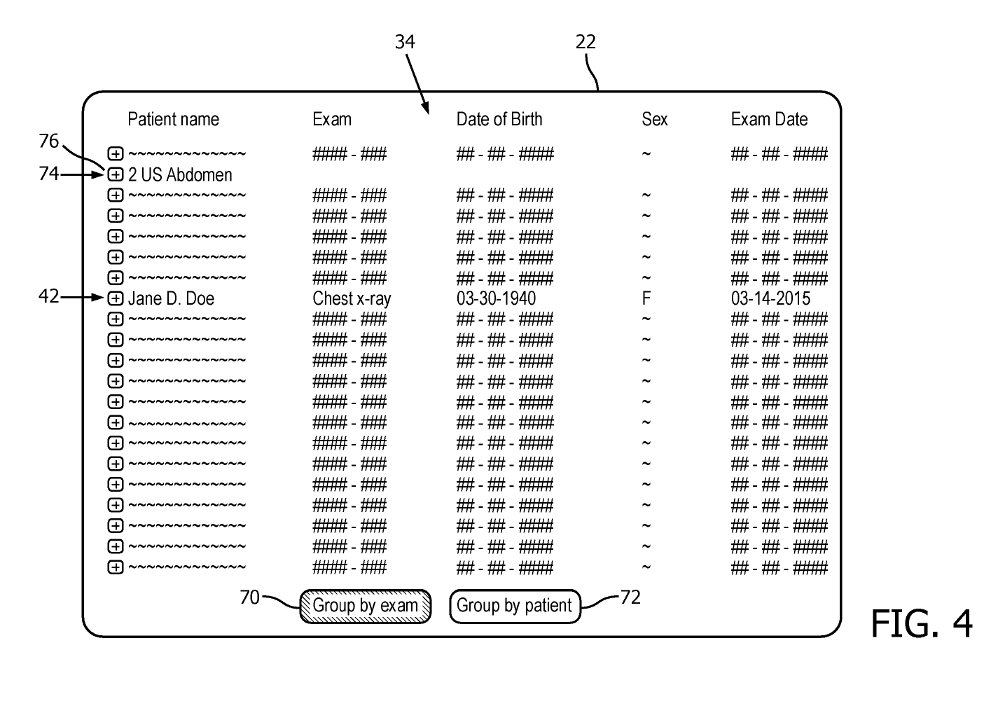

FIG. 4 illustrates the result, for the indicated reading tasks 40, 42, 44, when the radiologist selects button 70 to group by examination (type). As shown in FIG. 3, two radiology examination reading tasks 40, 44 are both “US Abdomen” (i.e., Abdomen Ultrasound) examinations. These are therefore grouped together as a task group 74 labeled “2 US Abdomen” in the work list 34 shown in FIG. 4. The remaining fields are not shown for task group 74, since they have different values for the different reading tasks of the task group 74. By pressing the expansion icon 76, the radiologist can expand the task group 74 to show the individual reading tasks 40, 44 (expansion not shown in FIG. 4, but sequentially lists entries 40, 44 shown in FIG. 3).

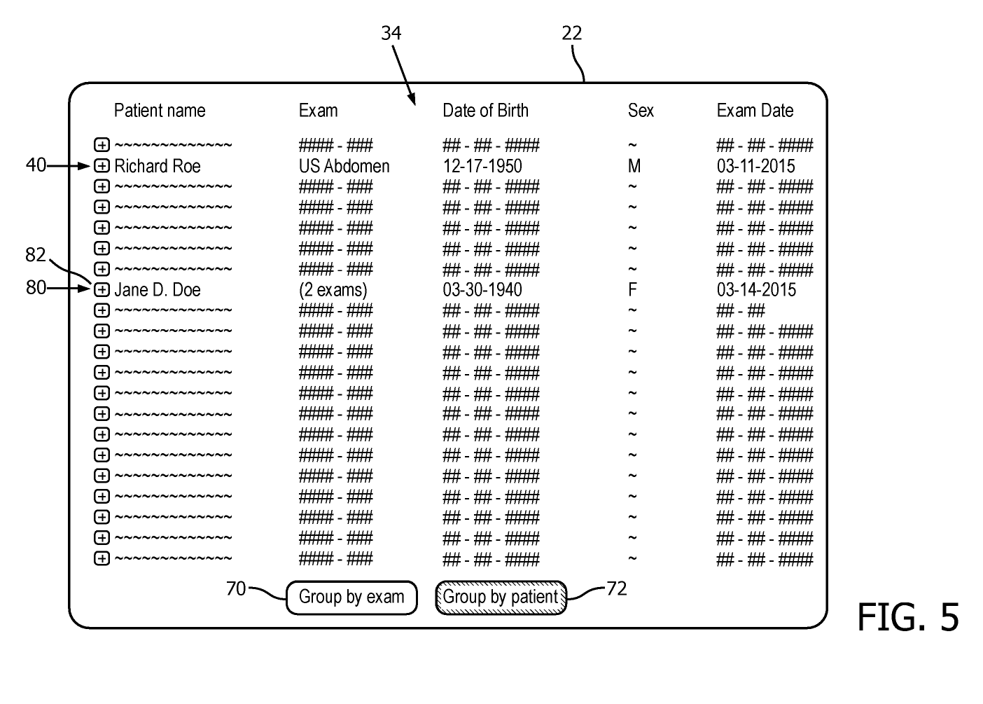

As another example, FIG. 5 illustrates the work list 34 generated for the case in which the radiologist presses the button 72 in order to group by patient. In this case, two reading tasks 42, 44 are for the same patient, namely “Jane D. Doe” accordingly, in the work list 34 shown in FIG. 5, these two reading tasks are grouped as patient group 80 containing all reading tasks for the patient “Jane D. Doe”. In this case, the date-of-birth and sex fields have their displayed values since these are specific to “Jane D. Doe”. The examination date field is not shown since the two examinations under the patient group 80 have different examination dates. Under the “Exam” field is listed “(2 exams)” in order to indicate the number of reading tasks contained in the patient group 80. By pressing the expansion icon 82, the radiologist can expand the patient group 80 to show the individual reading tasks 42, 44 (expansion not shown in FIG. 4, but sequentially lists the entries 42, 44 shown in FIG. 3).

In the embodiments just described with reference to FIGS. 3-5, the work list 34 is not tied to any particular reading schedule, but nonetheless assists the radiologist in selecting the next reading task to perform by grouping reading tasks that are advantageously performed together.

Time can be saved by reducing context-switching (mental change) of: the patient; the modality and body part; modality or body part. In the case of the example of FIG. 4, the radiologist benefits by grouping together reading tasks for the same type of examination because by performing these tasks together the radiologist reduces context-shifting between tasks. In the case of the example of FIG. 5, the radiologist benefits by grouping together reading tasks for the same patient because this facilitates developing a holistic view of the patient in which insights from one examination reading may inform another examination reading.

With reference to FIGS. 1 and 4, to perform the examination grouping of FIG. 4 the electronic processor 36 applies the rule 30A to identify groups of reading tasks of the same examination type (modality and anatomy). This grouping information is used by the search module 64 to construct examination groups, and the task ordering module 66 then constructs the work list 34 which groups reading tasks together by examination type (where possible).

With reference to FIGS. 1 and 5, to perform the patient grouping of FIG. 5 the electronic processor 36 applies the rule 30B to identify groups of reading tasks for the same patient. This grouping information is used by the search module 64 to construct patient groups, and the task ordering module 66 then constructs the work list 34 which groups reading tasks together by patient (where possible).

With returning reference to FIGS. 1 and 2, in embodiments that employ the reading schedule 50, rather than having the radiologist construct the reading schedule 50, e.g. using the schedule editor 50E, in other embodiments the electronic processor 36 is further programmed to implement a schedule trainer 50T that performs data mining of training data, comprising historical reading times 84 as a function of time-of-day, to construct the reading schedule 50 to allocate time blocks 52 in accordance with time intervals of high historical radiologist efficiency.

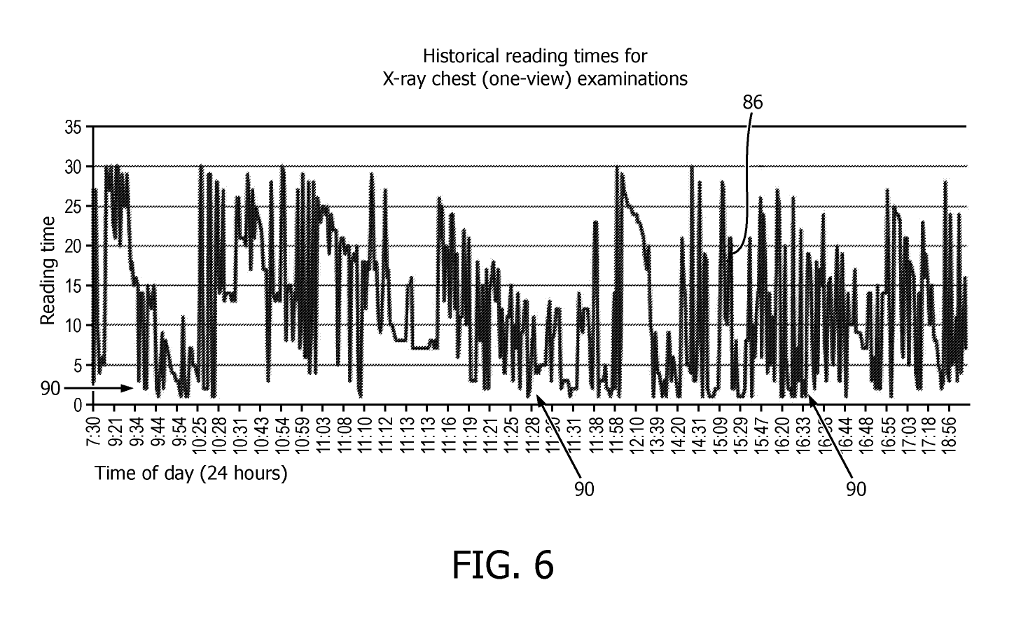

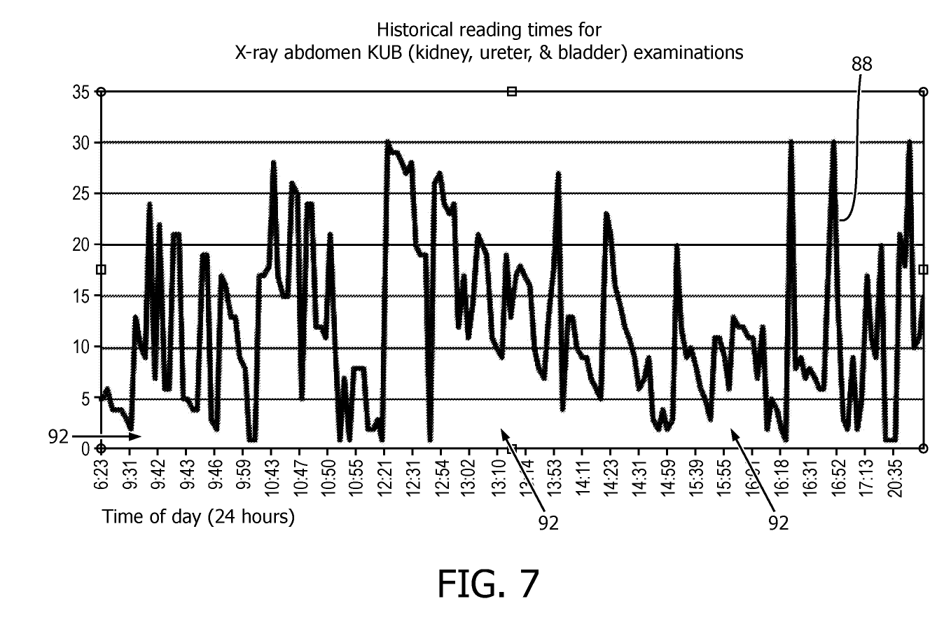

With reference to FIGS. 6 and 7, the training data may, for example, include a history of reading times for examinations of a particular examination type. FIG. 6 plots historical data comprising reading times 86 as a function of time of day for X-ray chest (one-view) examinations. FIG. 7 plots reading times 88 for X-ray abdomen KUB (kidney, ureter, & bladder) examinations. In general, the radiologist is exhibiting higher efficiency when the reading times are low. On this basis one or more “hot zones” 90, 92 are determined by the processor 36. (Other criteria can be used to assess radiologist efficiency, such as time blocks in which the radiologist generates the highest RVU points per unit time.) In FIG. 6, the hot zones 90 for X-ray chest examinations are those chest X-ray reading tasks performed in less than 5 minutes, i.e. the data below the “5 minute” on the y-axis of the graph of FIG. 6. Hot zones are thus observed at the 9:20-10:20 time block; the 11:15-12:00 time block; and the 12:30-4:45 time block. These hot zones 90 represent the time intervals that the radiologist has been, historically, most efficient at reading chest X-ray images. Accordingly, the schedule trainer 50T can advantageously allocate these time intervals of the reading schedule for performing chest X-ray reading tasks.

FIG. 7 shows data relating to an Abdomen KUB X-ray reading. As shown in FIG. 7, hot zones 92 are located at the 9:30-10 time block, the 10:45-12:30 time block; and the 4-4:15 time block. Accordingly, the schedule trainer 50T can advantageously allocate these time intervals of the reading schedule for performing abdomen KUB X-ray readings.

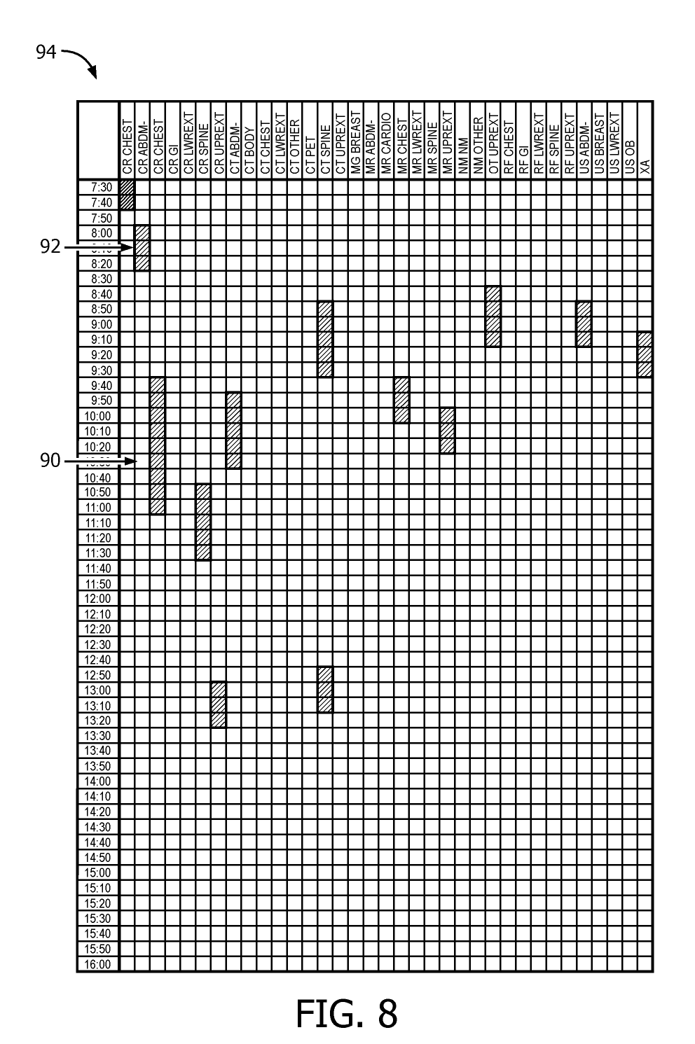

With reference to FIG. 8, based on these hot zones 90, 92 identified from historical reading times, a reading schedule 94 can be automatically generated. The schedule 94 shows allocated hot zones 90, 92 to optimize the RVUs of the radiologist during a given day for multiple types of image modalities. In constructing the reading schedule 94, where different examination types have overlapping hot zones the allocation of time blocks of the reading schedule can be variously handled. In one approach, the time block may be assigned for a mixture of the reading tasks having hot zones in that time block. In another approach, the relative number of examinations of each type may be taken into account in allocating time blocks, e.g. a more common examination type is assigned a larger time block.

Naturally, time-of-day hot zones can be made more specific by considering day-of-week. This is especially true when the week-long intensity may cause the radiologist to begin to tire slightly by the end of week.

It will be appreciated that the illustrative computational components may be embodied as a non-transitory storage medium storing instructions executable by an electronic processor (e.g. the workstation computer 16, or the PACS server 12) to perform the disclosed computations. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A device operating in conjunction with a Picture Archiving and Communication System (PACS) for storing a plurality of radiological images, the plurality of radiological images including at least two different image modalities, the device comprising:
a radiology workstation including at least one display device; and
an electronic processor programmed to:
organize a queue of radiology examination reading tasks in accord with information about the radiology examination reading tasks other than or in addition to their order in the queue to generate an ordered work list of the radiology examination reading tasks;
display the ordered work list on the at least one display device of the radiology workstation; and
retrieve from the PACS one or more radiology images of a radiology examination reading task of the ordered work list and display the retrieved radiology images on the at least one display device of the radiology workstation.

2. The device according to claim 1 wherein the radiology workstation is programmed to organize the queue to generate the ordered work list according to at least one rule the at least one rule including:
a rule grouping together radiology reading tasks in the ordered list by imaging modality;
a rule grouping together radiology reading tasks in the ordered list by imaging subject; and
a rule grouping together radiology reading tasks in the ordered list by imaging anatomy.

3. (canceled)

4. (canceled)

5. (canceled)

6. The device according to claim 1 wherein the radiology workstation is programmed to organize the queue to generate the ordered work list by assigning reading tasks to a time block of a reading schedule based on the reading tasks having a time block-defining feature;
wherein the time block-defining feature is an imaging modality, a radiology examination type, an imaged anatomy, or a combination thereof.

7. (canceled)

8. The device according to claim 6 wherein the radiology workstation is programmed to construct the reading schedule based on time intervals of high radiologist reading efficiency for reading tasks having time block-defining features identified in empirical radiologist reading time data.

9. The device according to claim 1 wherein the radiology workstation is programmed to organize the queue to generate the ordered work list including an ordered sequence of reading tasks following a pre-defined ordered sequence pattern.

10. (canceled)

11. The device according to claim 1 wherein the radiology workstation further includes:
at least one user input device, wherein the electronic processor is further programmed to receive a selection of a radiology examination reading task from the ordered work list via the at least one user input device, the retrieval operation being performed for the selected radiology examination reading task.

12. The device according to claim 1 wherein the radiology workstation further includes:
at least one user input device, wherein the electronic processor is further programmed to:
receive, via the at least one user input device, instruction to re-order radiology examination reading tasks of the ordered work list;
re-order the ordered work list in accord with the received instruction to generate an updated ordered work list; and
on the at least one display device, replace the ordered work list with the updated ordered work list.

13. The device according to claim 12 wherein the at least one user input device includes a pointing device or touch-sensitive display via which re-order instruction is received comprising a drag-and-drop operation in which one or more reading tasks are dragged and dropped to a different position in the ordered work list.

14. A device operating in conjunction with a Picture Archiving and Communication System (PACS) (IP), the device comprising:
a radiology workstation including at least one display device at least one user input device; and
an electronic processor programmed to:
retrieve a queue of radiology examination reading tasks in which the reading tasks are ordered by time of entry into the queue;
organize the queue of radiology examination reading tasks in accord with reading task features including or derived from at least one of imaging modality, imaged anatomy, radiology examination type, and examination subject to generate an ordered work list of the radiology examination reading tasks;
display the ordered work list on the at least one display device of the radiology workstation;
receive a selection of a radiology examination reading task from the ordered work list via the at least one user input device of the radiology workstation; and
retrieve from the PACS one or more radiology images of the selected radiology examination reading task and display the retrieved radiology images on the at least one display device of the radiology workstation.

15. The device according to claim 14 wherein the electronic processor is programmed to organize the queue of radiology examination reading tasks by grouping together in the ordered work list reading tasks having a common reading task feature.

16. The device according to claim 14 wherein the electronic processor is programmed to organize the queue of radiology examination reading tasks by grouping together in the ordered work list reading tasks having the same examination subject.

17. The device according to claim 14 wherein the electronic processor is programmed to organize the queue of radiology examination reading tasks by grouping together in the ordered work list reading tasks of the same imaging modality and the same imaged anatomy.

18. The device according to claim 14 wherein the electronic processor is programmed to organize the queue of radiology examination reading tasks by operations including:
constructing a reading schedule comprising time blocks defined by reading task features;
assigning reading tasks of the queue to time blocks in accord with the time block-defining reading task features to generate the ordered work list; and
with each time block, ordering the assigned reading tasks by time of entry into the queue.

19. (canceled)

20. The device according to claim 18 wherein the reading schedule is constructed based on time intervals of high radiologist reading efficiency for reading tasks with time block-defining reading task features identified in empirical radiologist reading time data.

21. The device according to claim 14 wherein the electronic processor is programmed to organize the queue to generate the ordered work list including an ordered sequence of reading tasks following an ordered sequence pattern defined by reading task features.
